(19) United States
(12) Patent Application Publication
(10) Pub. No.: US 2026/0102098 A1
(43) Pub. Date: Apr. 16, 2026

(54) SYSTEMS AND METHODS FOR MAPPING ARRYTHMIA OR FIBRILLATION

(71) Applicant: COREMAP, INC., Burlington, MA (US)

(72) Inventor: Peter S. Spector, Burlington, MA (US)

(21) Appl. No.: 19/357,763

(22) Filed: Oct. 14, 2025

Related U.S. Application Data
(60) Provisional application No. 63/707,393, filed on Oct. 15, 2024.

(51) Int. Cl. A61B 5/287; A61B 5/00; A61B 5/263; A61B 5/339; A61B 5/361; A61B 5/367; A61M 25/01

(52) U.S. Cl. CPC A61B 5/287; A61B 5/339; A61B 5/361; A61B 5/367; A61B 5/6852; A61M 25/0147; A61B 5/263; A61M 2205/0216; A61M 2205/0266

(57) ABSTRACT

The invention provides an asymmetrically flexible cardiac mapping catheter that includes a deformation mechanism by which a user may deform a distal portion of the catheter giving the user the ability to reach, and map electrical activity of, substantially any surface within the heart.

### TECHNICAL FIELD

The disclosure relates to systems and methods for mapping arrythmia or fibrillation in the heart.

### BACKGROUND

Atrial fibrillation is an arrhythmia of the heart that is concerning due to its increasing prevalence and high economic burden. Tissue ablation is one treatment approach. In patients with chronic atrial fibrillation, success-rates of ablation treatments are low. Treatment is thought to be difficult due to issues with multiple atrial wavelets, macro-reentries, and localized sources of atrial fibrillation (AF) referred to as drivers. Additionally, ablating to create lesions in a heart, while the heart is beating, is challenging and requires both 3D navigation and mapping of electrical activity with both high spatial resolution and near-complete coverage over cardiac tissue.

Conventional mapping systems may not fully detect localized AF drivers due to their spatiotemporal limits. For example, a chamber of the heart presents a complex surface in 3D space that varies in time and from case-to-case. Conventional mapping systems may not have the resolution, reach, and flexibility to be useful map all activity within a chamber of the heart. Due to those limits, some localized drivers of AF may not be fully and correctly mapped by conventional system.

### SUMMARY OF THE INVENTION

The invention provides a cardiac mapping catheter with electrodes on an asymmetrically-flexible distal head. The catheter has a mechanism by which a user may, from a proximal portion of the catheter, deform or bend the distal head in in either of at least two different directions and bend the distal head into a curve with a radius of curvature as low as 12 mm. This allows the head of the catheter to measure electrical activity at just about any location within a chamber of a heart via the electrodes. The deformation mechanism may include some form of switch or actuator (e.g., joystick, scroll-wheel, lever(s), or directional pad) at a proximal portion of the catheter, allowing the head to be bent into different curved shapes while the head is being used within the chamber of the heart. The head may include positioning indicators, such as magnetic coils or imaging-opaque fiducial markers, allowing a connected system to track a position and orientation of the head and electrodes in three-dimensions.

Because the electrodes are measuring electrical activity from the surface in the chamber of the heart, the system can build up a 3D model of the chamber of the heart, with a map of the mapped electrical activity depicted as part of the 3D model. Because the deformation mechanism allows the head to be bent in different directions, to a very tight radius of curvature, electrical activity of the heart may be measured and mapped at substantially every location within the chamber. The head may include an electrode array with a high number of electrodes (e.g., 44, or even 44 electrode pairs), giving the catheter a very high spatial resolution in mapping. The catheter maps electrical activity over substantially every location within the chamber with high spatial resolution, allowing the system to present a comprehensive and detailed 3D model of electrical activity of the heart. Due to the comprehensive and detailed 3D model, a surgeon may identify and location stable drivers of atrial fibrillation. By those means, the asymmetrically flexible catheter, with its user manipulable deformation mechanism, provides a tool useful to guide ablation for the treatment of atrial fibrillation.

In certain aspects, the invention provides a cardiac mapping catheter or system that includes the catheter. The catheter includes an elongated catheter body having a proximal portion and a distal portion, a head comprising an electrode array connected to the distal portion of the catheter body through a neck portion, a deformation mechanism operable to deform the distal portion to position an electrode of the electrode array at substantially any location on a surface in a chamber of a heart; and at least one conductive lead (e.g., 88 or more leads or wires) extending from the electrode to the proximal portion of the catheter body. The distal portion of the catheter body may be bent to a radius of about twelve mm in at least one direction. Preferably, the distal portion of the catheter body is capable of bending in a first direction and a second direction opposed to the first direction.

Any suitable deformation mechanism may be included. In some embodiments, the deformation mechanism has one or more tension wires extending from the head of the catheter and through the catheter body to a control mechanism the proximal portion of the cardiac mapping catheter. The distal portion is deformed by a user manipulating the proximal portion.

The catheter may be included in a system in which the proximal portion is connected to a processing device and/or a computer system. For example, the processing device may, in turn, be coupled to a computer system comprising at least one microprocessor coupled to a memory subsystem and a display device. The computer system may be operable to use measurements made from the head of the catheter to display, on the display device, a model of a heart in which the electrode array takes measurements. The processing device may include one or more of a multiplexer, an amplifier, and an analog-to-digital converter.

In some embodiments, certain signal processing is performed at the head of the catheter. The head of the catheter have at least about forty electrodes (or electrode pairs, e.g., at least 80 electrodes). The head of the catheter may include at least one chip to perform the functionality of a multiplexer, an amplifier, or an analog-to-digital converter. The electrode array may be connected to the chip, e.g., so that 40 or more wires extend from the head to the chip, where the chip is located in the head of a neck connecting the head to the catheter body. A smaller number of wires (e.g., just a few) may extend from the chip, through the catheter body, and to the processing device and/or computer system

In related aspects, the invention provides a method of for mapping arrythmia or fibrillation in a heart. The method includes: (i) navigating a distal portion of a catheter into a chamber of heart, in which the distal portion of the catheter carries a head comprising a deformable leaf of material with at least one electrode on a first a surface of the deformable leaf, (ii) operating a deformation mechanism of the catheter by manipulating an actuator connected to a proximal portion of the catheter to thereby deform the deformable leaf so that the first surface exhibits a first curve in a first direction, (iii) controlling the actuator to deform the leaf to cause the first surface to exhibit a second curve in a second direction opposed to the first direction; and (iv) measuring electrical activity of tissue of the heart while the first surface has the first curve and while the first surface has the second curve. Preferably, one or both of the first curve and the second curve have a radius of curvature no greater than about twelve mm.

The head may include an array of at least 30 electrodes (e.g., 40, optionally at least 40 stacked electrode pairs, thus at least 80 electrodes), in which each electrode is connected via a wire to a processing chip. The deformation mechanism may include at least an anterior tension wire and a posterior tension wire extending from the head to an actuator (such as a switch, joystick, a scroll wheel, a directional pad, or lever) at the proximal portion of the catheter, wherein the actuator is operable to pull the anterior tension wire to deform the deformable leaf so that the first surface exhibits the first curve.

In certain embodiments, a proximal portion of the catheter is coupled to a processing device and at least about thirty wires pass from the at least thirty electrodes through a body of the catheter the processing chip in the processing device. In embodiments with on-catheter signal processing, at least about thirty wires pass from the at least thirty electrodes through the processing chip in the distal portion of catheter, and fewer than thirty signal wires pass from the processing chip, through a body of the catheter, and to a processing device or computer system connected to a proximal portion of the catheter.

The deformation mechanism allows the at least one electrode to make measurements at substantially any location within a chamber of the heart. The head of the catheter may include a deformable leaf (e.g., of a polymer material) carrying the electrodes. The catheter may extend through an included sheath. When the catheter is pulled in a proximal direction into the sheath, the deformable leaf may fold into a substantially cylindrical configuration and when the catheter extends out from the sheath, the deformable leaf may open into a substantially open configuration. The deformable leaf may include a shape-memory or elastic support frame (e.g., of a nickel-titanium alloy or other elastic material) attached thereto, wherein the support frame biases the deformable leaf into the substantially open configuration.

### BRIEF DESCRIPTION OF DRAWINGS

FIG. 1 shows a system for mapping arrythmia or fibrillation.

FIG. 2 gives a close-up view of the head carrying an electrode array.

FIG. 3 is a detail view of an electrode of the electrode array.

FIG. 4 shows a cardiac mapping catheter with a deformation mechanism.

FIG. 5 shows the result of operating a deformation mechanism of the catheter.

FIG. 6 shows controlling the actuator to deform the catheter.

FIG. 7 shows the catheter inserted into, and mapping, a chamber of a heart.

FIG. 8 shows using the catheter in the heart.

FIG. 9 shows the catheter in the heart deformed in a second direction.

FIG. 10 illustrates an optional, e.g. bedside, processing device or workstation.

FIG. 11 illustrates a microprocessor unit integrated into a catheter of the disclosure.

FIG. 12 shows a display of the disclosure.

### DETAILED DESCRIPTION

Cardiac mapping systems of the disclosure provide intra-cardiac mapping catheters with distal portions that are asymmetrically flexible catheters and that optimal radius of curvature in at least one direction (e.g., down to 12 mm) and thus an optimal reach. The distal portion presents a head with a plurality of electrode (e.g., at least 30, preferably more than 40 and those may be stacked electrode pairs, so that the head includes at least 80 electrodes). Each electrode is at the terminus of its own signal wire so catheters of the disclosure have a large number of conductive wires, or leads, being handled in the head and—is some embodiments—extending through a neck of, and the body of, the catheter to be connected to signal processing system to which the catheter is attached. Specific embodiments have been made that include 44 stacked electrode pairs, with 88 associated wires (as well as positioning sensor wires) passing through neck and body while including a deformation mechanism that bends the head in two directions with bends exhibiting a radius of curvature as low as 12 mm. Related embodiments include at least one signal processing chip at the head or neck of the catheter, so that fewer than the e.g., 88 wires pass through the body. Catheters of the invention are preferable coupled via an optional processing device to a connected computer system e.g., that provides a real-time display to guide navigation & ablation.

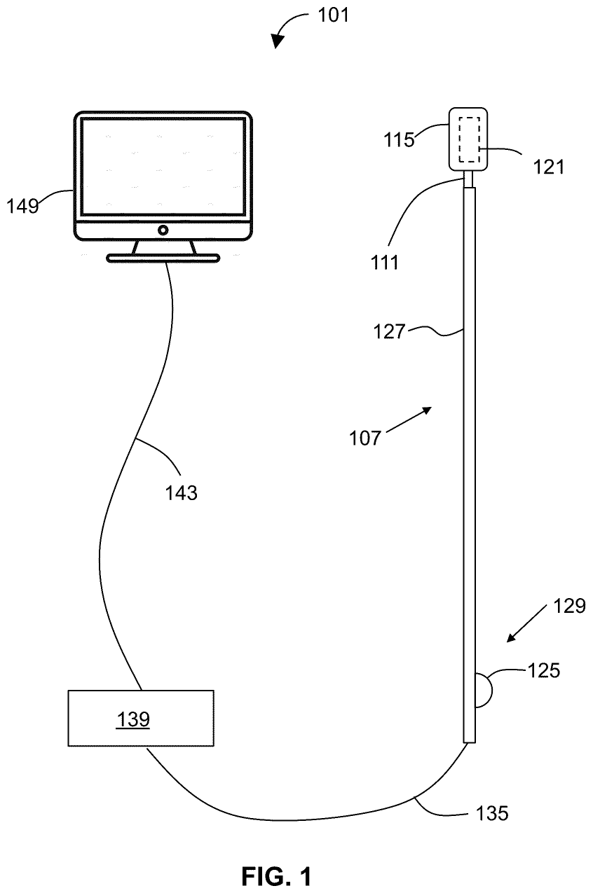

FIG. 1 shows a system 101 for mapping arrythmia or fibrillation. The system 101 includes a cardiac mapping catheter 107 of the invention. The cardiac mapping catheter 107 may generally include a shaft 111 extending to a head 115 carrying an electrode array 121. Electrodes of the electrode array 121 may optionally be connected via leads or at least one signal wire 135 to an optional processing device 139, which in turn have a data connection 143 to an optional computer system 149. The extend shaft 111 may extend through, and out of a distal end of, an introducer sheath 127. The catheter 107 includes a deformation mechanism 129 that is operable to change a shape, position, or dimension of the head 115 to thereby position the electrode array 121 at substantially any position within the heart. Any suitable deformation mechanism 129 may be included. Preferred embodiments use a switch, controller, or actuator 125 positioned at, or communicatively coupled via, a proximal end of the shaft 111 or sheath 127, to be manipulated by a surgeon from outside of a body of a patient during use of electrode array 121 in the heart.

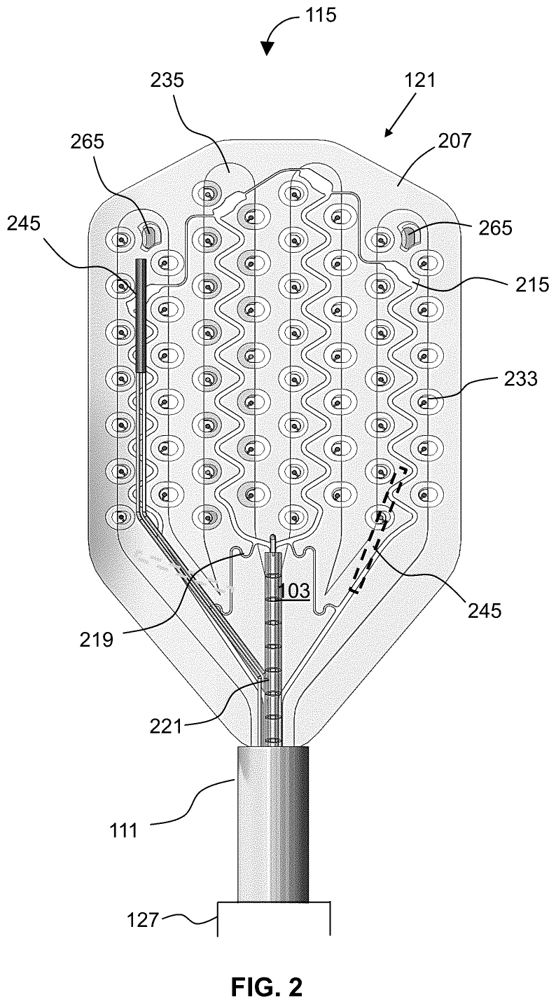

FIG. 2 gives a close-up view of the head 115 carrying an electrode array 121. In some embodiments, the head 115 includes a flexible support frame 215 extending within a deformable leaf 207 of a material, presenting the electrode array 121 on a tissue-facing surface. Due to the support frame 215, the head 115 may be substantially furled (or curled) closed, e.g., in a cylindrical configuration when the head is retracted within a sheath. When the head is extended out, in a distal direction, from a distal end of the sheet, the deformable leaf 207 of a material may assume a substantially flat, but “floppy”, aspect, allowing the electrode array 121 to conform to a shape of a tissue. Cardiac mapping catheters of the invention are capable of being reshaped when it is retracted or withdrawn into a sheath and unfurled when extended from a sheath. When held within a sheath, the electrode array 121 may be rolled or furled. When deployed from a sheath, the electrode array 121 unfurls to lie at least partially against the tissue upon which it is deployed. The flexible and deformable nature of the catheter ensures maximum flexibility to conform to the shape of the tissue upon which it is deployed thus allowing the catheter to lie flat against the tissue. Thus, the deformability and flexibility of the electrode array are enabled by the flexible support frame 215 and the deformable leaf 207.

The electrode array 121 generally includes a plurality of a sensing electrode 233 available on the tissue-facing surface of the deformable leaf 207. The support frame 215 may be made with shape memory and super-elastic material such as Nitinol, or other alloys of nickel and titanium. The support frame 215 may be designed so that when the head 115 within a sheath, the deformable leaf 207 is substantially s-shaped, rolled, or folded. After the electrode array is deployed from a sheath, it opens up to an unfurled configuration such that the electrode array 121 lies against the tissue upon which it is deployed to match the contours of the tissue.

The deformable leaf 107 may include one or more layers of the same or different materials. The deformable surface may be fabricated, for example, as two or more layers, such as by using a first material to develop first layer, then adding to it a second layer using a second material. The deformable surface may have a first layer having a first durometer number, and a second layer having a second durometer number lower than the first durometer number. The use of two materials of different durometers gives the catheter the flexible and bendable nature that allows it to conform to and adhere to the surface of the cardiac tissue thus improving the electrode contact with the tissue surface while also allowing the catheter tip to flatten easily after exiting a catheter sheath. For example, the deformable surface may include a first layer of a suitable non-metallic material such as polyimide, polyether ether ketone (PEEK, or nylon with the first layer having a higher durometer number than a second layer material, such as a thermoplastic polymer e.g., the polyamide and polyether block polymer sold under the trademark PEBAX by Arkema Global (Colombes, France). In another example, the deformable surface may have a first layer with, for example, PEEK or a thermoplastic polyurethane having a durometer number of 87D, and a second layer of a polyamide and polyether block polymer having a durometer number of 55D. The second layer material may then form a web around the first material. In some embodiments, the second layer may form a web-like structure around the first layer. The web structure may appear as open interstices or may resemble a web, netlike, or netted pattern.

The support frame 215 may be connected to the deformable leaf 107 on either side of the deformable leaf 107, or it may be retained within the deformable surface. In some embodiments, the cardiac mapping catheter comprises a deformable surface such that the catheter is undulated upon deployment from a sheath and lies flat when deployed against tissue.

The head 115 may include a plurality of tines 235 or splines extending from the catheter shaft through the electrode array. For example, the tines 235 may appear as arms or in the form of slender projecting or branching pieces to engage the tissue surface. The tines may be flat on both sides with a rounded edge. Each electrode 233 may be positioned along an edge of a tine 235, distributed over the head to form the electrode array 121. The tines 135 or splines may comprise a flexible printed circuit (FPC).

The catheter 107 includes a deformation mechanism 129 that is operable to deform the head 115. Any suitable deformation mechanism 129 may be included. Preferred embodiments use a first tension write 219 and a second tension wire 219 (not visible on the head 115, being on the reverse side). The tension wire 1219 may extend the length of the catheter body to the actuator 125. The wire 219 may be controllable and may be used to deform the surface of the catheter to match the surface of the cardiac tissue. By actively modifying the angle of the electrode array via the tension wires 219, the deformable leaf 107 may be bent in two different directions. In some embodiments, cardiac mapping catheters of the invention include a spine tube 221 in connection with the flexible support frame 215 of the electrode array 121 and a pull wire 219 also in connection with the flexible support frame. In some embodiments, the spine tube 221 is connected to the catheter shaft 101 and the tension wires 219, and housed within or upon the deformable leaf 107. The spine tube 221 may be notched to aid in deflection of the electrode array 121 once the array is deployed from a sheath. In some embodiments, the spine tube is actuated by a mechanism such as a handle or trigger (e.g., actuator 125) operably connected to the cardiac mapping catheter.

The tension wires 219 may be provided so that when the electrode array is deployed out of a sheath 127, the tension wires 219 allows the electrode array to deflect in either direction such that the physician can manipulate the electrode array to a desired position upon the tissue.

Catheters of the invention may include one or more location or position markers 245. Each position marker 245 may be a magnetic coils, (e.g., as magnetic tracking sensors or magnetic location sensors). The position markers 245 may be positioned on opposing sides and/or opposite ends of the deformable leaf 107. Having magnetic coils in the opposing positions allow for shape detection of the array and identification of each electrode's position in 3D space. The position markers 245 may provide a 5 degrees of freedom (DOF) sensor, wherein the degrees of freedom describe the number of axes in which a rigid body moves freely in 3D space. In some embodiments, data from the magnetic sensors is used to calculate the position of the electrode array 121 to model a shape of a surface from which measurements are made.

In some embodiments, the electrode array may further comprise a plurality of pacing electrodes 265, positioned for the measurement of a refractory period and/or to stimulate activation of a cardiac rhythm. Thus, in some embodiments of the invention, the cardiac mapping catheters of the invention allow for sensing, mapping, and pacing functions on the single device.

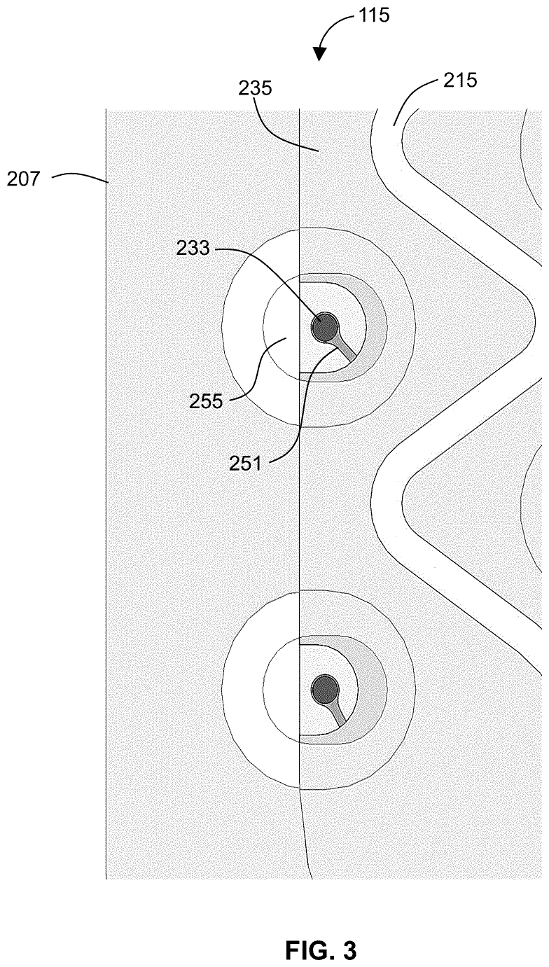

FIG. 3 is a detail view of an electrode 233 of the electrode array 121, here viewed from a tissue-facing (or “posterior”) surface of the deformable leaf 207. Portions of the support frame 215 and at least one edge of one tine 235 are visible. Preferably, the electrodes 233 are present in an ordered array, with pairs of the electrodes 233 being separated by a known distance. Each electrode 233 as shown may, in fact, be a single electrode, or each electrode as shown may include a pair of electrode contact elements that are stacked. Each electrode 233 may comprise two or more materials. For example, the electrodes may include an iridium oxide coating 203 surrounded by a layer or region of insulating material 255.

In some embodiments, individual sensing electrodes 133 include a copper trace 251 and a copper pad that may be plated with gold. The gold may cover the copper components of the electrode to prevent the copper from leaching into solution. The electrode may then be further coated with iridium oxide to provide a layer of iridium oxide to reduce the impedance and to improve signal-to-noise ratio. In some embodiments, the electrode comprises a slot below the insulating layer into which the copper trace is embedded such that the gold plating is on top of the copper electrode.

Each electrode 233 may include electrode contact elements shaped substantially as thin discs. The electrodes, as thin discs, may be positioned as a contiguous array on the surface of the deformable surface. The electrodes may be positioned along an edge of a tine 235 or spline. Notably, the height, or alternatively the thickness, of the electrode may be less than about150 μm. In some embodiments, the height of the electrode is less than about 25 μm. Height, as used herein, means the height of the electrode as measured from the bottom of the electrode relative to its orthogonal position on the tissue to the top of the electrode. Alternatively, the height of the electrode may also refer to the thickness of the electrode as a thin disc.

In some embodiments, the electrode array comprises a plurality of openings configured to allow biological fluid and/or a wash fluid such as saline to pass through the head 115. For example, the openings may be formed through the leaf 207.

In non-limiting embodiments, the diameter of each electrode 233 may be 150 μm such that the electrode surface area is less than or equal to 0.018 mm\hat{ }2. The electrode array may comprise a number (e.g., 30, 40, 44, 50, 56 . . . ) electrodes on each side of the deformable surface. The electrode array width may be less than or equal to 15 mm, with an overall length less than or equal to 26 mm. The array sensing area may be less than or equal to 15 mm\hat{ }2. The cardiac mapping catheter may be used with a sheath 127, such as an 8.5F steerable sheath with a diameter ranging from about 0.122 inches to about 2.83 mm.

Electrodes may be unipolar, bipolar, or of a hybrid arrangement involving a stacked pair of bipolar electrodes that provides advantages of unipolar electrodes. For conventional electrophysiological procedures, intra-cardiac electrodes have been deployed in two distinct configurations, unipolar and bipolar. For bipolar recordings, a different electrode is positioned at the site of interest while the indifferent electrode is positioned close to, but spaced away from, the different one. Wave fronts that pass the electrodes induce similar signals at both poles, but with the signals being shifted in time. In the unipolar recording mode, a different electrode is positioned at the site where the electrical potential must be determined while the indifferent electrode is positioned a large distance from the heart, at zero potential. Unipolar electrodes are adequate but have a tendency to include far field electrical activity in the recorded signal which can result in a fractionated electrogram, of concern when trying to map complex arrhythmias (e.g. cardiac fibrillation) and accurately identifying local activation time. Bipolar electrode configurations minimize that fractionation by placing both electrodes within the heart at a relatively narrow distance apart. Both electrodes of a bipolar pair measure approximately the same far field electrical activity with the result that the resultant electrogram includes little far field signal. Here, catheters of the disclosure may use another electrode configuration comprising pairs of electrodes that are, in fact, bipolar but in which the pair is designed to sit in a stack over one location of the tissue so that, like with unipolar electrodes, signal recorded by the stacked electrode pair tends to reflect electrical activity from all regions of the heart even when the stacked electrode pair is placed directly over a particular region. Thus, each electrode may be provided as, or as part of, an electrode pair having a “stacked” or orthogonal close unipolar (OCU) electrode configuration. This electrode array may include a two dimensional array of stacked electrode pairs (e.g., first electrode and second electrode layered with insulator therebetween). The first electrode is configured to be close to a location on a surface of target tissue and the second electrode is separated from the first electrode but over the same location. Each electrode pair may be provided in an orthogonal, close, unipolar (OCU) configuration. Of the pair, the two electrodes are stacked such that when the leaf 207 is against tissue, an axis through the stack of the two electrodes is substantially orthogonal to a surface of the tissue where the electrode pair is making a reading (“orthogonal” nature of the pair). A distance between the first and second electrodes is preferably about a same order of magnitude as a diameter or thickness of each electrodes so that the members of the pair are close to each other (“close” nature of the pair). The one pair of bipolar electrodes may operate to measure electrical activity from all regions of the heart as expected from unipolar electrodes (“unipolar”nature of the pair).

Such a configuration, referred to as an OCU configuration, addresses the limitations of existing unipolar and bipolar electrodes. In particular, recorded electrical potential of current bipolar electrodes vary with their orientation relative to the direction of a passing wavefront. Additionally, because bipolar electrodes have both electrodes on a given surface, there is potential inclusion of distinctly different electrical activity from each electrode. As such, by providing electrodes oriented perpendicular to the tissue plane, via the orthogonal close unipolar (OCU) design of the present invention, the electrode array of the present disclosure retains the superior near/far-field discrimination of common bipolar electrode recordings with the directional independence and smaller footprint of unipolar recordings. Furthermore, the unipolar electrode configuration of the present invention retains all of the spatial resolution benefits of a contact bipolar configuration, but with the additional spatial resolution enhancement conferred by a smaller footprint.

Embodiments of the disclosed cardiac mapping catheter may include unipolar or bipolar electrode configurations. Preferred embodiments include OCU configurations. The electrodes 233 preferably form an electrode array on the head 115 connected to the distal portion of the catheter 107, in which the catheter includes a deformation mechanism operable to deform the distal portion to position an electrode 233 of the electrode array at substantially any location on a cardiac surface.

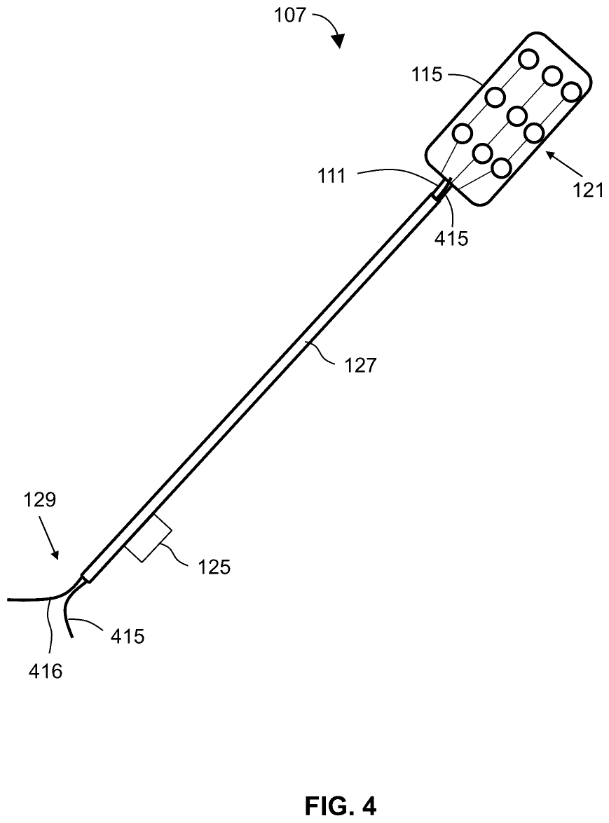

FIG. 4 illustrates one embodiment of a cardiac mapping catheter 107 with a deformation mechanism 406. As shown, the shaft 111 extends to a head 115 carrying an electrode array 121. The shaft 111 extends from an introducer sheath 127. The catheter 107 includes a deformation mechanism 129 that is operable to change a shape, position, or dimension of the head 115 to thereby position the electrode array 121 at substantially any position within the heart. Any suitable deformation mechanism 129 may be included. Preferred embodiments use a switch, controller, or actuator 125 positioned at, or communicatively coupled via, a proximal end of the shaft 111 or sheath 127, to be manipulated by a surgeon from outside of a body of a patient during use of electrode array 121 in the heart. The deformation mechanism 129 allows for a user to exhibit control over the head 115 of the catheter 107 to reach any cardiac surface. A cardiac surface is any portion of the heart. This includes exterior portions such as the base, the diaphragmatic surface, the sternocostal surface, and left and right pulmonary surfaces; this also includes internal surfaces such as the walls of the atria and ventricles as well as the septum and valves. The deformation mechanism 129 may use a tension wire 415 that, when pulled in a proximal direction via a surgeons manipulation of the actuator 125, causes the head 115 (and thus the electrode array 121) to curl inwards in a posterior (in the direction that the electrodes face) direction. As shown, the deformation mechanism 129 includes a posterior tension wire 415 and an anterior tension wire that may be manipulated to deform the head 115 in two different directions, asymmetrically, off-axis from the shaft 111. The tension wires 415, 416 of the deformation mechanism 129 may comprises a metal, polymer, or any other suitable material. The tension wires 415, 416 may further have coatings e.g., of an anticoagulant, Teflon, silicone, or any other suitable material. In some embodiments, the deformation mechanism 129 is operated electronically. The deformation mechanism 129 may be configured to work with electronic or robotic controls to assist the user in guiding the catheter throughout the patient.

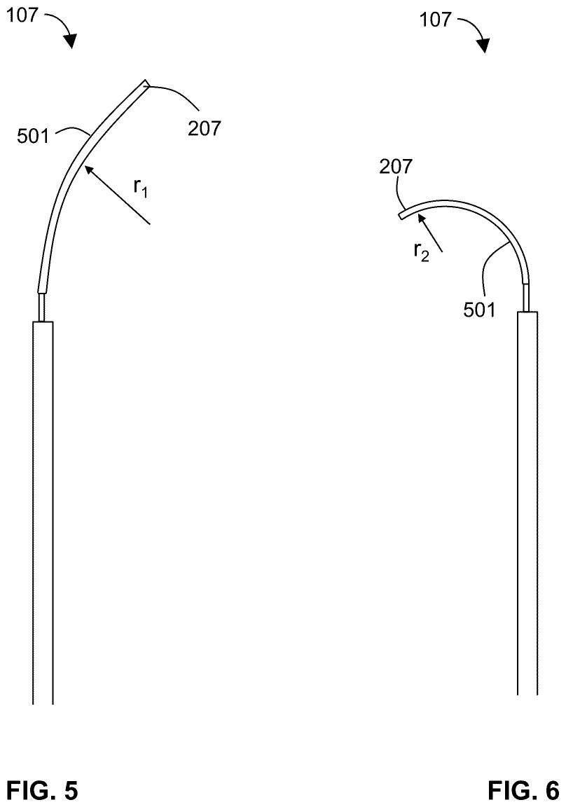

FIG. 5 shows the result of operating a deformation mechanism of the catheter 107. Operation of the mechanism has deformed the deformable leaf 207 so that a first surface 501 exhibits a first curve in a first direction. Using the same deformation mechanism, on may curve the first surface 501 in an opposed, second direction.

FIG. 6 shows the result of controlling the actuator 125 to deform the leaf 207 to cause the first surface 501 to exhibit a second curve in a second direction opposed to the first direction. As shown, the first curve has a radius of curvature r1. The second curve has a radius of curvature r2. The catheter preferably carries at least about 30 (preferably at least 40, e.g., 44 or more) OCU electrode pairs, meaning that the head includes at least 60 (e.g., at least 88) electrodes, and the catheter may include 60 or more (e.g., 88 or more) conductive wires extending from the leaf 207 to a proximal portion of the catheter 107. The catheter 107 may be used for measuring electrical activity of tissue of the heart while the first surface has the first curve and while the first surface has the second curve. One or both of the first curve and the second curve have a radius of curvature such that r1 and/or r2 are no greater than about 12 mm.

As shown, the invention provides an asymmetrically flexible cardiac mapping catheter that includes a deformation mechanism by which a user may deform a distal portion of the catheter giving the user the ability to reach, and map electrical activity of, substantially any surface within the heart.

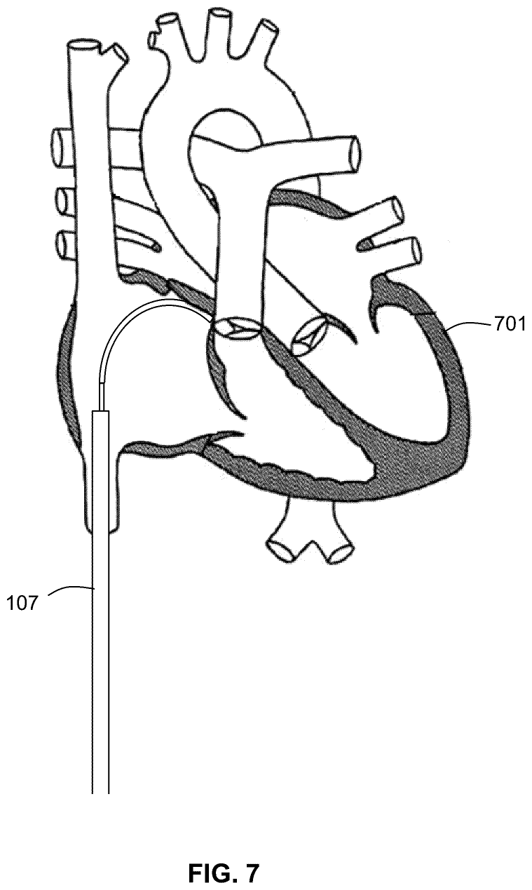

FIG. 7 shows the catheter inserted into a chamber of a heart 701, and being used to map electrical activity of the heart 701. Using the disclosed deformation mechanism, substantially any location on a surface of a chamber of the heart 701 may be mapped by the catheter 107. By deforming the leaf 207 in a first direction, and then in a second direction, and by bending the leaf 207 to a very small (e.g., 12 mm) radius cover curvature, an electrode 233 (or OCU pair) on the leaf 207 may take a reading at substantially any location within the chamber.

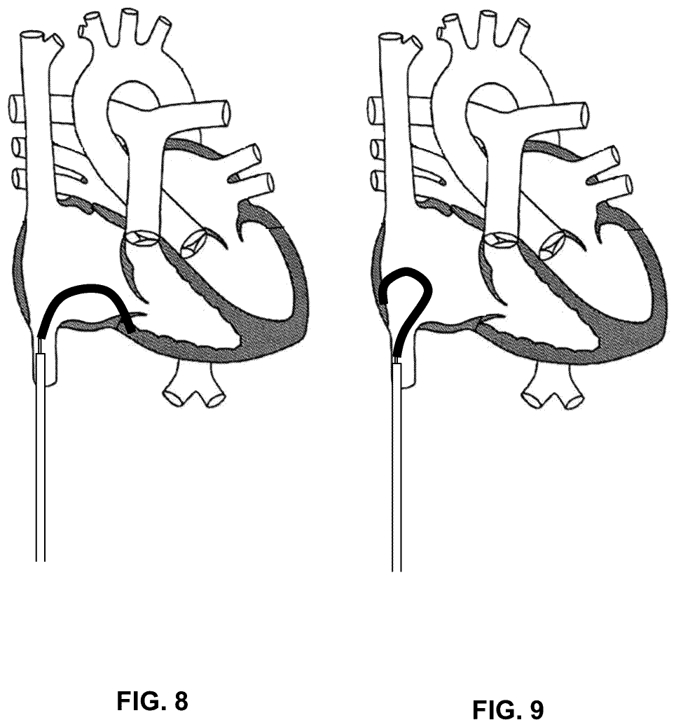

FIG. 8 shows using the catheter 107 in the heart 701 with the deformable leaf 207 deformed in a first direction to take measurements at a location.

FIG. 8 shows using the catheter 107 in the heart 701 with the deformable leaf 207 deformed in a second direction (opposed to the first direction) to take measurements at another location. Because the distal head has a radius of curvature as low as 12 mm, the head of the catheter may be used to measure electrical activity at just about any location within a chamber of the heart via the electrodes.

Other functions and features may be included with devices, systems, and methods of the invention. For example, circuitry among the electrode array 121 or processing performed by the optionally connected processing device 139 and/or computer system 149 may perform a common mode rejection (CMR) operation.

A CMR operation may improve the spatial resolution of electrogram recordings by decreasing the size of a region of cardiac tissue that contributes to the electrogram recording. The CMR electrode array configuration is able to accurately measure a signal derived exclusively from activation as it passes beneath a central electrode; it eliminates contribution to the local signal by activation of the tissue surrounding a central electrode.

A “central” electrode in the array that detects the local activation signal works in conjunction with multiple “surrounding” contact electrodes that surround the central electrode on the array. It is noted that numerous or any of the various electrodes 233 may each, in turn, be a central electrode for CMR, and while one electrode 233 is being treated as central, the other electrodes that surround it are treated as the surrounding electrodes. As a local signal passes underneath the central electrode, a signal is concurrently recorded from both the central electrode and the surrounding electrodes. The signals from the surrounding electrodes are averaged, and the resulting average is subtracted from the central electrode signal. As a result, the CMR electrode array is able to accurately measure a given signal (e.g., an activation signal when used in cardiac tissue) by eliminating both far-field signal interference and near-field signal interreference from other electrodes on the array. As a result, a signal detected by the central electrode represents only the signal generated by the activation signal as it passes underneath the electrode. The CMR electrode array is thus able to leverage the benefits of unipolar electrodes and bipolar electrodes, while eliminating their drawbacks.

Accordingly, the use of simultaneously obtained electrode data according to the invention is useful to determine relative positions of measurements made by multiple electrodes in the construction of cardiac tissue mapping. A CMR may allow or aid in the computation of conduction velocity. The array can be used to construct a map of cardiac rhythm and tissue properties, such as scarring.

As discussed above, a catheter may be connected to a processing device 139.

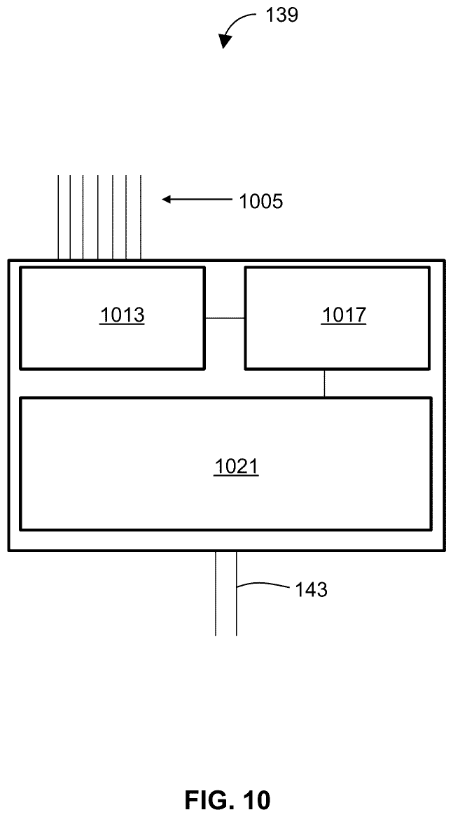

FIG. 10 illustrates the optional processing device 139. The processing device 139 is preferably a special-purpose device built specifically to perform signal processing on electrical readings made via the electrodes 233 of the electrode array 121. The processing device 139 may be a hardware unit standing bed-side in a catheterization laboratory (“cath lab”) or hospital or clinical facility. The processing unit may mediate signals between the electrodes array 121 and a connected computer system 149 (which commonly in a cath lab may not be in the same room and may use cloud or server computers that are remote (other city or state) to a control computer terminal in an observation room of the cath lab). The processing device 139 may receive a plurality of conductive leads 1005 which extend from the electrodes 233 mounted on the head 115 of the catheter 107. The processing device 139 may include one or more of a multiplexer 1013, an amplifier 1017, and an analog-to-digital (ADC) converter 1021 (in any order). The processing device 139 may further include a data connection 143 (e.g., one or more wires) to transmit the converted signal to a computer system 149.

In one embodiment, when a signal is transmitted through the conductive leads 1005, it arrives at the multiplexer 1013. In the apparatus of the present invention, 80 or more conductive leads carrying signals from 80 or more different electrodes can arrive at the multiplexer 1013. The multiplexer 1013 is a device that selects between several analog or digital input signals and forwards the selected input to a single output line. A multiplexer makes it possible for several input signals to share one device or resource, for example, one analog-to-digital converter or one communications transmission medium, instead of having one device per input signal. The catheter of the present invention may optionally utilize a demultiplexer, which is a device that takes a single input and selects signals of the output of the compatible multiplexer, which is connected to the single input, and a shared selection line. A multiplexer is often used with a complementary demultiplexer on the receiving end. The multiplexer 1013 of the present invention makes it possible for the plurality of conductive leads 1005, which can often comprise 80 or more wires, to be paired down to a significantly lower number of wires.

In certain embodiments, an analog multiplexer may be used. An analog multiplexer interprets a continuously variable signal form multiple input sources. A digital multiplexer may also be used. Digital multiplexers interpret signals that usually take only finite levels. For example, an analog signal may be represented by a wave while a digital signal may be represented by a series of 1s and 0s.

After a signal travels through the conductive leads 1005 and the multiplexer 1013, it arrives at the amplifier 1017. An amplifier is an electronic device that can increase the magnitude of a signal (a time-varying voltage or current). It is a two-port electronic circuit that uses electric power from a power supply to increase the amplitude (magnitude of the voltage or current) of a signal applied to its input terminals, producing a proportionally greater amplitude signal at its output. The amount of amplification provided by an amplifier is measured by its gain: the ratio of output voltage, current, or power to input. An amplifier is defined as a circuit that has a power gain greater than one. An amplifier can be either a separate piece of equipment or an electrical circuit contained within another device. Amplifiers can be categorized in different ways. One is by the frequency of the electronic signal being amplified.

The amplifier 1017 can be more than one type of amplifier. Depending on its placement within the signal chain, the amplifier could be a preamplifier. A preamplifier oftentimes comes first in a signal processing chain and converts a weak signal into a stronger one for further processing. The amplifier 1017 may also be a linear amplifier. A linear amplifier is an electronic circuit whose output is proportional to its input, but is capable of delivering more power into a load. Linearity refers to the ability of the amplifier to produce signals that are accurate copies of the input. A linear amplifier responds to different frequency components independently and tends not to generate harmonic distortion or intermodulation distortion. No amplifier can provide perfect linearity however, because the amplifying devices follow nonlinear transfer function and rely on circuitry techniques to reduce those effects. The amplifier 1017 may also be a transimpedance amplifier. A transimpedance amplifier is a current to voltage converter, often implemented with one or more operational amplifiers. A transimpedance amplifier converts the low-level current of a sensor to a voltage however, the voltage is proportional to the input current.

Following amplification of the signal, the signal is transmitted to the analog-to-digital converter 1021 (ADC). An ADC is a system that converts an analog signal to a digital signal. Once the signal has been processed via the processing device 139, it is transmitted through the catheter body and to the computer system 149 via the data connection 213.

Other embodiments are within the scope of the disclosure. For example, in some embodiments, at least some of the signal processing is initially performed on the catheter 107 itself, e.g., by one or more chips that may be installed at the head 115. In certain embodiments, the head 115 of the catheter includes at least one chip that provides a multiplexer, an amplifier, or an analog-to-digital converter and wherein the electrode array is connected to the chip.

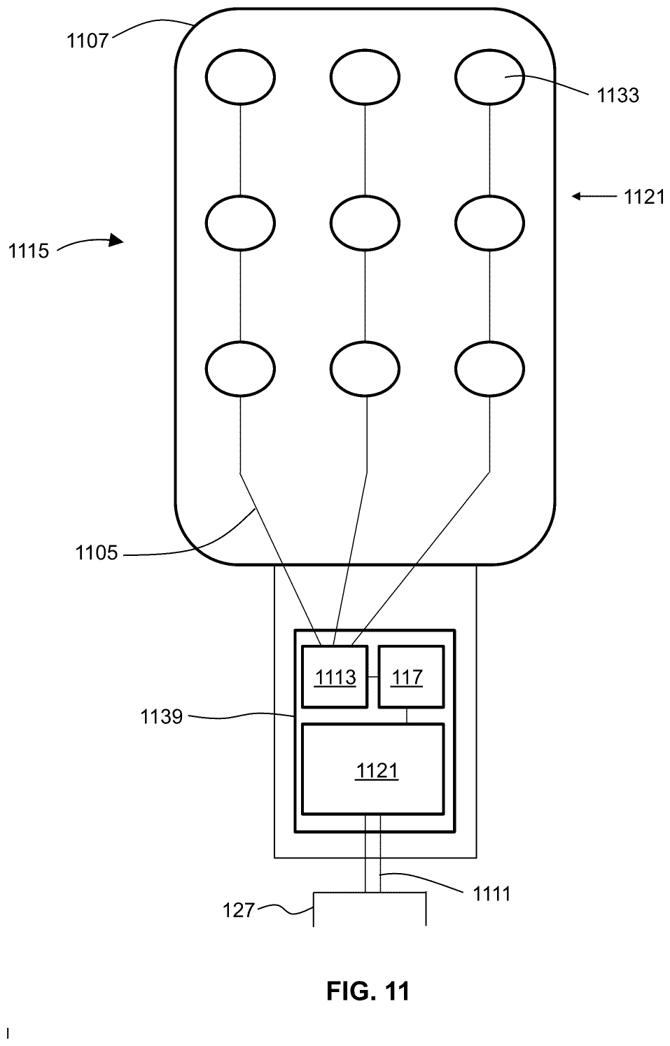

FIG. 11 illustrates a microprocessor unit 1139 integrated with a distal head 1115 of a catheter of the disclosure. The head carries at least electrodes 1133, preferably as part of an electrode array 1121. Each electrode 1133 is preferably available on the tissue-facing surface of a deformable leaf 1107. The head 1115 includes, connected thereto, a microprocessor unit 1139. As shown, the microprocessor unit 1139 may receive a plurality of conductive leads 1105 which extend from the electrodes 1133 mounted on the head 1115 of the catheter. The processing device 1139 may include one or more of a multiplexer 1113, an amplifier 1117, and an analog-to-digital (ADC) converter 1121 (in any order). The processing device 1139 may further include a data connection 143 (e.g., one or more wires or fibers) to transmit signals along shaft 1111 (through sheath 127) to a connected computer system 149 (optionally via an extracorporeal processing device 139).

Whether via the optional processing device 139, the onboard microprocessor unit 1139, or a combination of both, catheter of the disclosure provide signals representing electrical activity of the heart to the connected computer system 149.

The computer system 149 preferably includes at least one processor coupled to a memory subsystem containing instructions therein executable by the processor to cause the computer system 149 to perform methods of the disclosure. The computer system 149 may optionally any suitable hardware such as the processor coupled to random access memory (RAM) and a non-transitory, computer-readable storage system, a graphics card, a sound card, and/or input/output (I/O) devices such as a mouse, a keyboard, speakers, data interconnects, monitors or other displays, and network connections. Preferably, the computer system 149 is operable to display visual images representing patient anatomy and physiology based on the signals and data received from using the catheter.

The visual data may be based in part on data obtained before or during the procedure. The visual data on the computer screen may update in dynamically, in real time. Data obtained prior to the procedure may also be used in conjunction with real-time date. Data obtained prior to the procedure may include any type of (two-dimensional or three-dimensional) medical imaging including, but not limited to, computed tomography scans (CT scans), X-rays, magnetic resonance imaging (MRI), ultrasound, elastography, tomography, echocardiograms, and the like. Optionally, visual data obtained during the procedure may be superimposed on previously obtained medical images and displayed on the computer system 149. The computer system 149 may also process other types of data, in addition to visual data, such as electrical data.

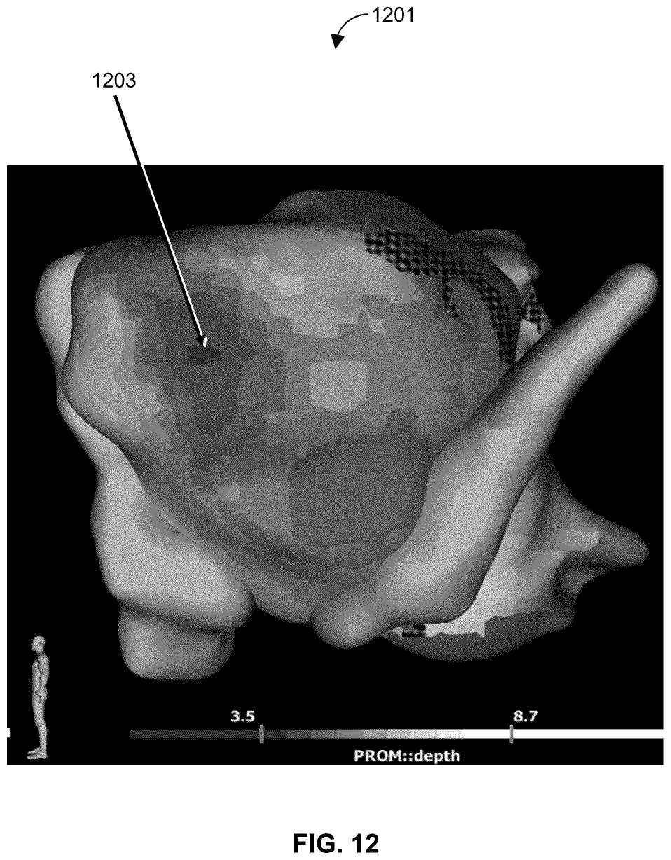

FIG. 12 shows a display 1201 that may be presented by the computer system 149 to show a map of the electrical activity of a heart 701 (here, superimposed on a model of the heart 701) that was created by using the catheter 107 to map the heart 701.

What is claimed is

1. A cardiac mapping catheter comprising: an elongated catheter body having a proximal portion and a distal portion; a head comprising an electrode array connected to the distal portion of the catheter body through a neck portion; a deformation mechanism operable to deform the distal portion to position an electrode of the electrode array at substantially any location on a cardiac surface; and at least one conductive lead extending from the electrode to the proximal portion of the catheter body.

2. The cardiac mapping catheter of claim 1, wherein the distal portion of the catheter body can bend to a radius of about 12mm in at least one direction.

3. The cardiac mapping catheter of claim 1, wherein the distal portion of the catheter body is capable of bending in a first direction and a second direction opposed to the first direction.

4. The cardiac mapping catheter of claim 1, wherein the deformation mechanism comprises one or more tension wires extending from the head of the catheter and through the catheter body to a control mechanism the proximal portion of the cardiac mapping catheter.

5. The cardiac mapping catheter of claim 1, wherein the distal portion is manipulated by a user at the proximal portion.

6. The cardiac mapping catheter of claim 1, wherein the proximal portion is connected to a processing device and the processing device is coupled to a computer system comprising at least one microprocessor coupled to a memory subsystem and a display device.

7. The cardiac mapping catheter of claim 6, wherein the computer system is operable to use measurements made from the head of the catheter to display, on the display device, a model of a heart in which the electrode array takes measurements.

8. The cardiac mapping catheter of claim 1, wherein the head of the catheter comprises at least about 40 electrodes.

9. The cardiac mapping catheter of claim 6, wherein the processing device includes one or more of a multiplexer, an amplifier, and an analog-to-digital converter.

10. The cardiac mapping catheter of claim 1, wherein the head of the catheter includes at least one chip that provides a multiplexer, an amplifier, or an analog-to-digital converter and wherein the electrode array is connected to the chip.

11. A method of for mapping arrythmia or fibrillation in a heart, the method comprising: navigating a distal portion of a catheter into a chamber of heart, wherein the distal portion of the catheter carries a head comprising a deformable leaf of material with at least one electrode on a first a surface of the deformable leaf; operating a deformation mechanism of the catheter by manipulating an actuator connected to a proximal portion of the catheter to thereby deform the deformable leaf so that the first surface exhibits a first curve in a first direction; controlling the actuator to deform the leaf to cause the first surface to exhibit a second curve in a second direction opposed to the first direction; and measuring electrical activity of tissue of the heart while the first surface has the first curve and while the first surface has the second curve.

12. The method of claim 11, wherein one or both of the first curve and the second curve have a radius of curvature no greater than about 12 mm.

13. The method of claim 11, wherein the head comprises an array of at least 30 electrodes, wherein each electrode is connected via a wire to a processing chip.

14. The method of claim 13, wherein the deformation mechanism comprises at least an anterior tension wire and a posterior tension wire extending from the head to an actuator at the proximal portion of the catheter, wherein the actuator is operable to pull the anterior tension wire to deform the deformable leaf so that the first surface exhibits the first curve.

15. The method of claim 14, wherein the actuator mechanism comprises a scroll wheel or lever.

16. The method of claim 14, wherein the proximal portion of the catheter is coupled to a processing device and wherein at least about 30 wires pass from the at least 30 electrodes through a body of the catheter the processing chip in the processing device.

17. The method of claim 14, wherein at least about 30 wires pass from the at least 30 electrodes through the processing chip in the distal portion of catheter, and wherein fewer than 30 signal wires pass from the processing chip, through a body of the catheter, and to a processing device or computer system connected to a proximal portion of the catheter.

18. The method of claim 11, wherein the deformation mechanism allows the at least one electrode to make measurements at substantially any location within a chamber of the heart.

19. The method of claim 11, wherein the catheter extends through a sheath and wherein, when the catheter is pulled in a proximal direction into the sheath, the deformable leaf folds within a substantially cylindrical configuration and when the catheter extends out from the sheath, the deformable leaf opens into a substantially open configuration.

20. The method of claim 11, wherein the deformable leaf has a shape-memory or elastic support frame attached thereto, wherein the support frame biases the deformable leaf into the substantially open configuration.
